(19) United States
(12) Patent Application Publication
(10) Pub. No.: US 2014/0163377 A1
(43) Pub. Date: Jun. 12, 2014

(54) Registration Using Phased Array Ultrasound

(71) Applicant: MAKO SURGICAL CORPORATION, Fort Lauderdale, FL (US)

(72) Inventors: Hyosig Kang, Weston, FL (US); Scott David Nortman, Sunrise, FL (US)

(73) Assignee: MAKO Surgical Corporation, Fort Lauderdale, FL (US)

(21) Appl. No.: 13/710,955

(22) Filed: Dec. 11, 2012

(51) Int. Cl. A61B 8/00

(52) U.S. Cl. CPC A61B 8/4488
USPC 600/447

(57) ABSTRACT

A system and method for performing registration of a bone structure to align a computerized model of the bone structure with the detected surface of the actual bone structure utilizes a two-dimensional ultrasound array. The array is positioned in a manner such that the position of the array is known relative to the actual bone structure. The array is phased to produce a point-focused beam of acoustic energy which is scanned in three dimensions throughout the volume of interest to map the surface of the bone structure.

### TECHNICAL FIELD OF THE DISCLOSURE

The present disclosure relates to computer-assisted surgery and robotically-assisted surgery and, more particularly, relates to a system and method for performing registration of a bone structure to align a computerized model of the bone structure with the actual bone structure.

### BACKGROUND OF THE DISCLOSURE

When performing computer-assisted surgery (CAS) and/or robotically-assisted surgery (RAS), it is often desirable to perform a registration step to align the computerized bone model with the actual patient bone. Existing systems achieve this goal by measuring point locations on the physical bone surface with a calibrated probe and feeding the point locations to the CAS/RAS system. Alternate attempted methods of measuring bone surface points have been investigated and include structured light vision systems and laser displacement sensors. A common drawback of all of the foregoing approaches includes the need to collect intra-incision data, requiring the surgeon to spend significant critical time on this cumbersome process.

Alternately, other bone surface sensing techniques are possible, in the inventor's view, to eliminate the intra-incision data collection requirement. One such technique includes ultrasound. Ultrasound point collection probes typically work in one of two ways: they either collect data from a single point, or collect data from a line. While both probing approaches may be promising, a significant problem still exists when collecting data. Specifically, both techniques require a probe to be dragged across the skin while maintaining acoustically sufficient contact and perpendicularity to image the bone surface. These requirements are difficult to implement and may make these types of probes too cumbersome for bone-to-model registration.

Due to these drawbacks, a need still exists that will allow for ultrasound based registration that eliminates the need to drag a probe across the skin. The present disclosure is directed to systems and methods that address one or more of the problems set forth above. However, it should be appreciated that the solution of any particular problem is not a limitation on the scope of this disclosure or of the attached claims except to the extent expressly noted. Additionally, the inclusion of any problem or solution in this Background section is not an indication that the problem or solution represents known prior art except as otherwise expressly noted.

### SUMMARY OF THE DISCLOSURE

In accordance with one aspect of the present disclosure, a method is provided for registration of a bone structure of a patient to align a bone structure model with the bone structure. The method entails placing a two-dimensional ultrasound transducer array adjacent the bone structure and performing an ultrasound scan of the bone structure with the two-dimensional ultrasound transducer array without substantial movement of the two-dimensional ultrasound transducer array relative to the bone structure to identify a surface of the bone structure. A bone model is then registered to the surface of the bone structure.

In accordance with another aspect of the present disclosure, a system is provided for registration of a bone structure of a patient to align a bone structure model with the bone structure. The system includes a two-dimensional ultrasound transducer array and a kinematic mount affixed to the two-dimensional ultrasound transducer array. A disposable acoustic gel pad is interposed between the two-dimensional ultrasound transducer array and the skin of the patient adjacent the bone structure.

In accordance with yet another aspect of the present disclosure, a non-transitory computer-readable medium having stored thereon computer-executable instructions is provided for registration of a bone structure of a patient via a two-dimensional ultrasound transducer array. The computer-executable instructions include instructions to vary a phase of each of a plurality of elements in the two-dimensional ultrasound transducer array to steer and focus a beam of ultrasound energy as well as instructions to scan the focused beam of ultrasound energy in three dimensions through a volume including the bone structure. Also included are instructions to record echo energy while scanning the point focused beam to determine a surface of the bone structure and instructions to align a model of the bone structure with the determined surface of the bone structure.

Other features and advantages of the disclosed systems and principles will become apparent from reading the following detailed disclosure in conjunction with the included drawing figures.

### BRIEF DESCRIPTION OF THE DRAWINGS

FIG. 1 is a schematic diagram of a linear ultrasound transducer array during beam steering;

FIG. 2 is a schematic diagram of a linear ultrasound transducer array during beam focusing;

FIG. 3 is a schematic diagram of a 2D (two-dimensional) ultrasound transducer array during an example 3D (three-dimensional) scan;

FIG. 4 is a perspective view of a 2D ultrasound transducer array during an example 3D scan;

FIG. 5 is a schematic view of a system for positioning a 2D ultrasound transducer array for registration;

FIG. 6 is a flow chart showing a process of bone structure registration using a 2D ultrasound transducer array;

FIG. 7 is a perspective view of a surgical system usable within embodiments of the disclosed principles; and

FIG. 8 is a perspective view of a transducer array and associated components according to an embodiment of the disclosed principles.

### DETAILED DESCRIPTION OF THE DISCLOSURE

The present disclosure provides a system and method for performing registration of a bone structure to align a computerized model of the bone structure with the actual bone structure. In an embodiment, a single large scanning ultrasound probe remains substantially fixed on the skin while collecting bone surface points over an area of a target surface by steering and focusing of an ultrasound beam. In particular, rather than a point or a linear phased array of ultrasound transducers, the disclosed technique employs a two-dimensional (2D) array of transducers which emit acoustic pulses at precisely calculated intervals, effectively ‘steering’ and focusing the resultant acoustic wave energy in three dimensions over the area of registration. This new mode allows for full XY steering and depth control or focus of the acoustic energy, which allows registration of an underlying bone structure without dragging an ultrasound probe over a patient's skin.

The disclosed probe, also referred to as an XY Phased Array Registration Block, contains an XY array of ultrasound transducers in a single block along with a calibrated kinematic mount. The block attaches to a disposable acoustic gel pad, and is then affixed to the patient's skin above the target bone. Once placed onto the skin, there is no need to move the unit to collect the required registration points.

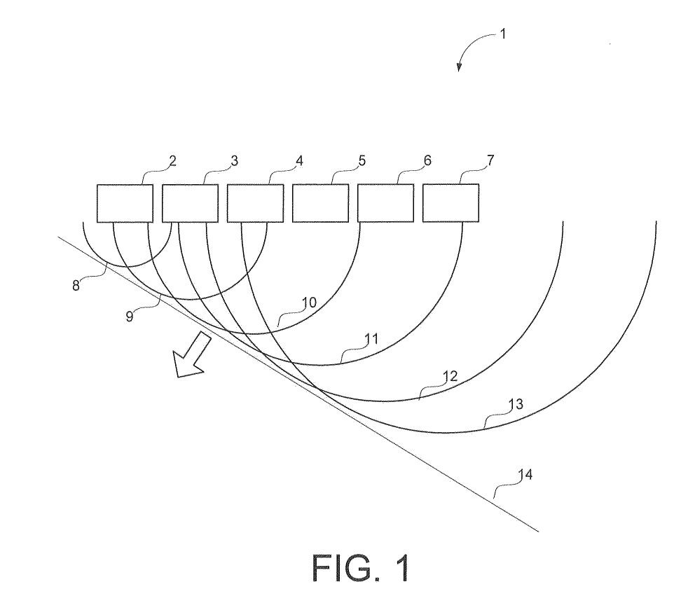

Turning to the figures, FIG. 1 is a schematic view of a traditional linear phased transducer array, showing wave propagation in an example transmission. As can be seen, the linear phased transducer array 1 includes a linear series of individual transducers 2, 3, 4, 5, 6, 7. In practice, a much greater number of transducers would be used. Each transducer 2, 3, 4, 5, 6, 7 is individually controlled to emit an ultrasonic wave pulse at a prescribed time, such that the emitted waves 8, 9, 10, 11, 12, 13 constructively interfere along line 14.

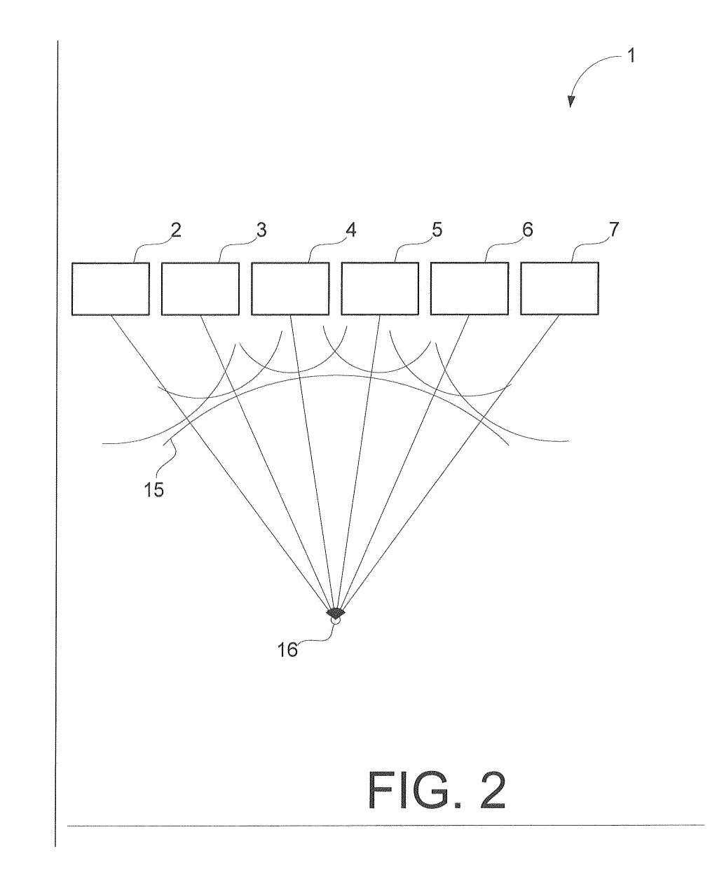

As the delay between the various emitted waves 8, 9, 10, 11, 12, 13 is changed, the exact location of the constructive interference can be scanned along the line 14 or placed at another location in the three-dimensional (3D) volume in front of the sensor array 1. FIG. 2 shows the linear phased transducer array 1 during focusing. In particular, the transducers 2, 3, 4, 5, 6, 7 are controlled to emit phased ultrasonic wave pulses such that the resultant constant-phase wave front 15 converges to a point of constructive interference at point 16. The timing of various transducers 2, 3, 4, 5, 6, 7 may be further delayed or accelerated to steer the beam, i.e., to move the location of the constructive interference. As such, the linear phased transducer array 1 is able to scan a 2D plane parallel to the array. However, as noted above, a linear probe such as this must be dragged along the patient's skin very carefully to produce a 3D image or registration of the entire bone surface of interest.

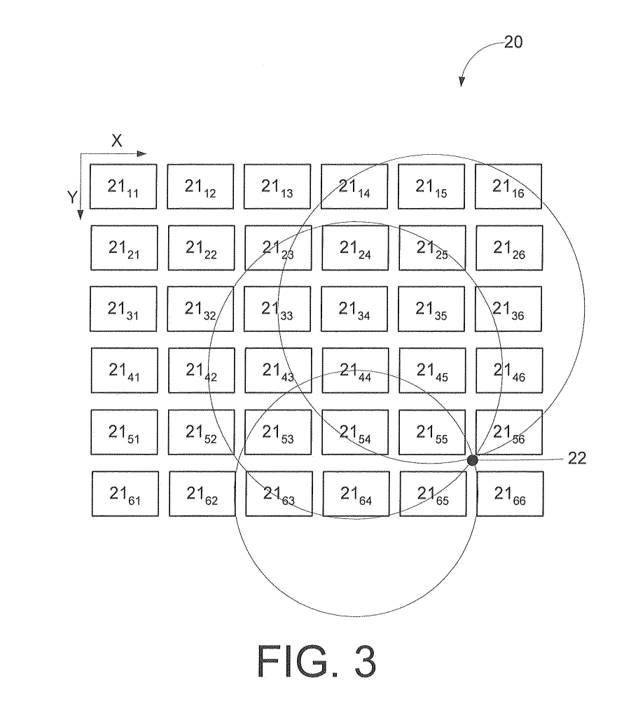

FIG. 3 is a schematic image of a 2D ultrasound transducer array 20 within which an embodiment of the disclosed principles may be implemented. The 2D ultrasound transducer array 20 includes a plurality of ultrasound transducers 21ₘₙ, where the subscripts m and n represent row and column respectively of a given transducer. In the illustrated embodiment, the direction of emission is toward the reader, while the direction of reception is toward the image.

Although all of the plurality of ultrasound transducers 21ₘₙ will typically be used during beam steering and focusing, a point of constructive interference 22 is shown based on only three of the plurality of ultrasound transducers 21ₘₙ for clarity. In particular, phased emissions from transducers 21₃₅, 21₄₄, and 21₆₄ are shown constructively interfering at point 22. It will be appreciated that the phase of the participating transducers may be varied to place the point of interference elsewhere as well, both in the XY plane (steering) and in distance from the array (focus). In this way, the ultrasound beam can be steered and focused rather than requiring the array itself to be moved during registration of a target bone area.

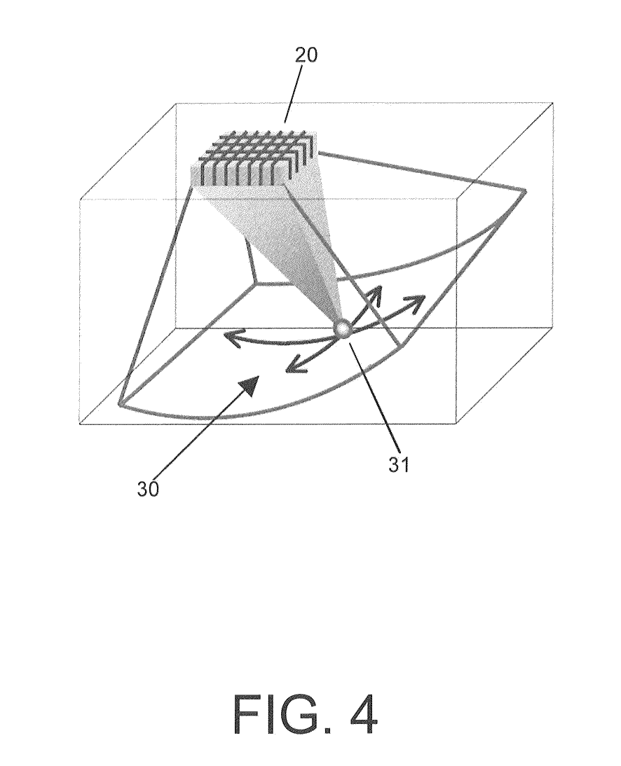

Thus it will be appreciated that in addition to scanning an ultrasonic beam in a manner generally parallel to the XY plane, the array 20 can also scan the point of focus of the ultrasonic beam in the direction perpendicular to the array. In this manner, the array 20 can be used to construct a 3D image of a patient's bone structure. This is illustrated schematically in FIG. 4, wherein the array 20 is used to produce a scan of a volume 30 via a steered and point-focused ultrasonic beam 31. The scan may scan over successive surfaces for example, building up reflection data from each scanned surface, without any movement of the array 20 on the patient's body.

In practice, the reflection data is referenced to the position and orientation of the array 20 relative to a marker (46) on the patient in order to register the bone model to the actual bone as tracked by the marker (46). As such, it is important to know with reasonable accuracy what the position and orientation of the array 20 are relative to the marker 46. In this way, the physical location of the bone structure relative to the frame of reference of the CAS or RAS system is known.

In an embodiment, an array of ultrasound transducers in a single block such as array 20 is attached to a calibrated kinematic mount. The block is attached to a disposable acoustic gel pad, which is then affixed to the skin above the target bone. The disposable acoustic gel pad provides good acoustic contact between the array and the skin. Once placed on the skin and registered with the marker, scanning (steering and focusing) takes place without movement of the block.

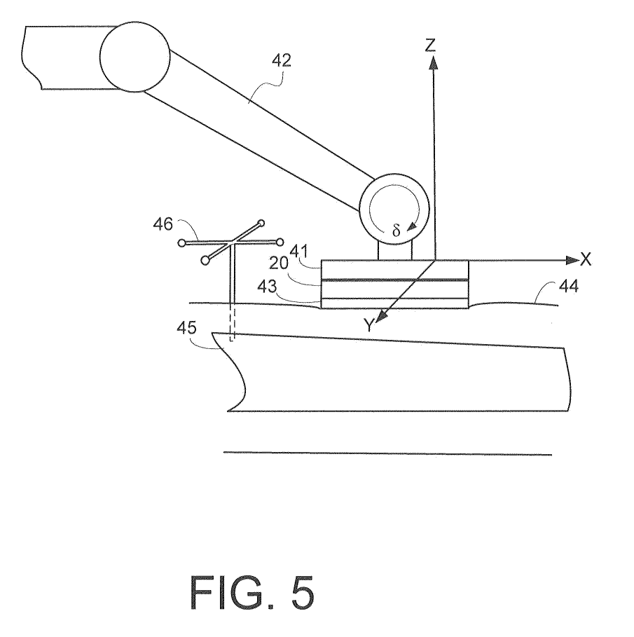

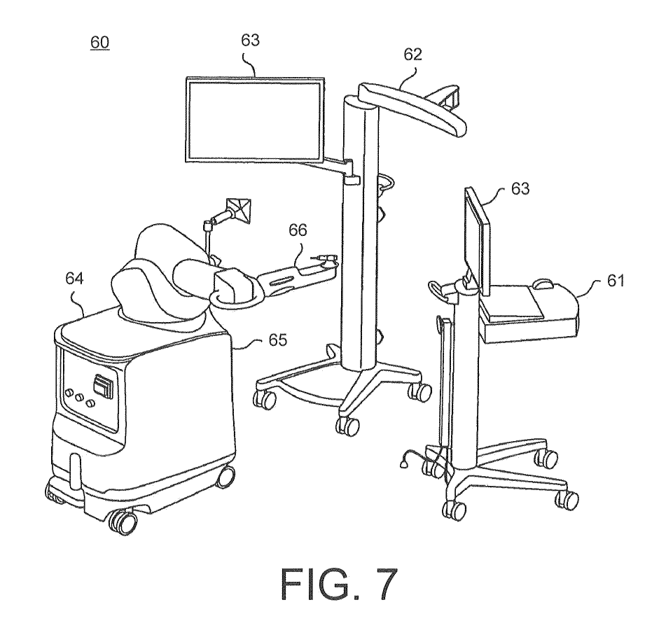

FIG. 5 shows the foregoing arrangement schematically. In particular, the array 20 is attached to the kinematic mount 41. In an embodiment, the position in the XYZ space and orientation δ of the kinematic mount are tracked via an optical or other marker affixed thereto. In an alternative embodiment, the position in XYZ space and orientation δ of the kinematic mount 41 are tracked via instrumentation associated with the mount and attached arm, e.g., angle encoders and other measurement devices. With respect to the latter embodiment, the kinematic mount 41 is attached to an articulated arm system 42 wherein the positions and orientations of the arms are measured by sensors. In an alternative embodiment, the kinematic mount 41 itself may be supplied with sensors, e.g., inertial and magnetic sensors, optical sensors, or other mechanism for tracking the movement of the mount such that the final pose of the mount 41 is known. It will be appreciated that the articulated arm system 42 may be a stand-alone system or may be a robotic arm system associated with a RAS/CAS system as discussed with respect to FIG. 7 further below.

In an embodiment, a disposable acoustic gel pad 43 is located between the array 20 and the patient's skin 44. This provides good acoustical contact and allows the array 20 to scan and locate the underlying bone structure 45. In order to track the location of the underlying bone structure 45 and to thus be able to register the bone structure 45 with a model based on the scanned data, a bone marker 46 may be affixed to the bone structure 45. The bone marker 46 may be adhered onto or into the bone structure 45 such that the bone structure 45 and the bone marker 46 move as one. The location of the bone marker 46 may be tracked by optical, magnetic, RF, or other means.

The array 20 is preferably connected to and controlled by a processor such as in a computer system, not shown. The processor operates by reading computer-executable instructions from a nontransitory computer-readable memory such as a RAM, ROM, flash drive, optical drive, and so on. The instructions dictate when an element in the array transmits in order to steer and focus the beam to sequential locations as the beam is scanned.

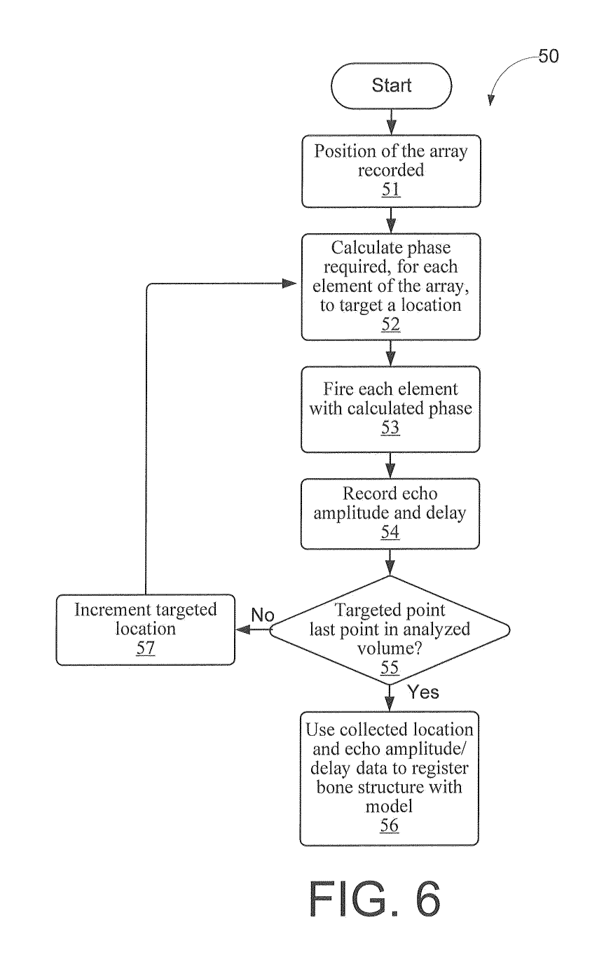

In keeping with the above, FIG. 6 is a flow chart showing a process of bone structure registration using a system such as that described above. The process 50 starts at stage 51, wherein the location and orientation of the array on the patient's skin, e.g., known via the kinematic mount or via interaction with a marker, is recorded relative to the location and orientation of the bone marker (46). At stage 52, the processor calculates the phase required for each element of the array 20 in order to target a location, e.g., point (x,y,z)=(a,b,c), in an analyzed volume.

The processor fires each element with the calculated phase at stage 53, and then records the echo amplitude and delay at stage 54. The echo amplitude and delay may be recorded in conjunction with the targeted location to produce a listing of associated locations and amplitudes/delays. At stage 55, the processor determines whether the targeted point was the last point in the analyzed volume.

If it is determined that the targeted point was the last point in the analyzed volume, the process 50 proceeds to stage 56, wherein the processor uses the collected amplitude and delay data to register the underlying bone structure with a model of the structure. For example, the processor may use the collected delay and amplitude data to identify a surface of the underlying bone structure. The identified surface may then be mapped, e.g., via a best fit or otherwise, to a model based on a prior imaging scan, such as a CT, MRI or other scan type known to those of skill in the art. Alternatively, the model may be one that is retrieved from a library of generic models and morphed to better fit the collected data. In an embodiment, the resultant model is displayed, but this need not be the case.

If instead it is determined at stage 55 that the targeted point was not the last point in the analyzed volume, the process proceeds to stage 57, wherein the processor increments the targeted location. For example, the scan of target locations may proceed along a series of horizontal lines or surfaces, vertical lines or surfaces, or may be interlaced or otherwise organized.

From stage 57, the process returns to stage 52, and the new target location is processed. It will be appreciated that the processor continues to track the location and orientation of the kinematic mount relative to the location and orientation of the bone marker (46).

Although the process 50 describes a method for registering a scanned structure without moving the array 20 itself during the scan, it will be appreciated that the described system may also be used to acquire multiple scans from different locations. In this embodiment, the scans may be registered to one another and stitched together for display if desired. Alternatively, the scans may be used as separate stand-alone scans.

In an embodiment, multiple scans from the same location may be used to increase accuracy. In particular, for example, repetitive scans of the same volume or same points within a volume may be averaged or otherwise combined to yield a higher accuracy scan of the volume or points of interest. This may be useful, for example, under conditions where slight unavoidable movements occur, etc.

Although any suitable RAS/CAS system may be used in conjunction with the described system and method, an exemplary system is shown in FIG. 7. The illustrated surgical robot system 60 includes a computer assisted navigation system 61, a tracking device 62, a display device 63 (or multiple display devices 63), and a robotic arm 64 as described, for example, in U.S. patent application Ser. No. 11/357,197 (Pub. No. US 2006/0142657), filed Feb. 21, 2006, which is herein incorporated by reference in its entirety.

The robotic arm 64 can be used in an interactive manner by a surgeon to perform a surgical procedure on a patient, such as a hip or knee replacement procedure. The robotic arm 64 includes a base 65, an articulated arm 66, an optional force system (not shown), and a controller (not shown). A surgical tool is coupled to the articulated arm 66, and the surgeon manipulates the surgical tool by grasping and manually moving the articulated arm 66 and/or the surgical tool. It will be appreciated that during acoustic scanning, the articulated arm 66 may be used to position the transducer array mounted on the kinematic mount in the same manner that the articulated arm system 42 is used.

The force system and controller may be configured to provide control or guidance to the surgeon during manipulation of the surgical tool. In particular, the force system may be configured to provide at least some force to the surgical tool via the articulated arm 66, and the controller may be programmed, in part, to generate control signals for controlling the force system. In one embodiment, the force system includes actuators and a backdriveable transmission that provide haptic (or force) feedback to constrain or inhibit the surgeon from manually moving the surgical tool beyond predefined virtual boundaries defined by haptic objects. In a preferred embodiment the surgical system is the RIO Robotic Arm Interactive Orthopedic System manufactured by MAKO Surgical Corp. of Fort Lauderdale, Fla. The force system and controller may be housed within the robotic arm 64.

The tracking device 62 is configured to track the relative locations of the surgical tool (coupled to the robotic arm 66) and the patient's anatomy. The surgical tool can be tracked directly by the tracking device 62. Alternatively, the pose of the surgical tool can be determined by tracking the location of the base 65 of the robotic arm 64 and calculating the pose of the surgical tool based on joint encoder data from joints of the robotic arm 64 and a known geometric relationship between the surgical tool and the robotic arm 64. In particular, the tracking device 62 (e.g., an optical, mechanical, electromagnetic, or other known tracking system) tracks (or enables determination of) the pose (i.e., position and orientation) of the surgical tool and the patient's anatomy so the navigation system 60 is apprised of the relative relationship between the tool and the anatomy.

In operation, an operator, e.g., a surgeon, manually moves the robotic arm 64 to manipulate the surgical tool to perform a surgical task on the patient, such as bone cutting. As the surgeon manipulates the tool, the tracking device 62 tracks the location of the surgical tool and the robotic arm 64 optionally provides haptic (or force) feedback to limit the surgeon's ability to move the tool beyond a predefined virtual boundary that is registered (or mapped) to the patient's anatomy, which results in highly accurate and repeatable bone cuts. The robotic arm 64 provides haptic feedback when the surgeon attempts to move the surgical tool beyond the virtual boundary. The haptic feedback is generated by one or more actuators (e.g., motors) in the robotic arm 64 and transmitted to the surgeon via a flexible transmission, such as a cable drive transmission. When the robotic arm 64 is not providing haptic feedback, the robotic arm 64 may be freely moveable by the surgeon and preferably includes a virtual brake that can be activated as desired by the surgeon.

During the surgical procedure, the navigation system 60 may display images related to the surgical procedure on one or both of the display devices 63. In an embodiment, the displayed images include an image generated via the volume scanning process described above.

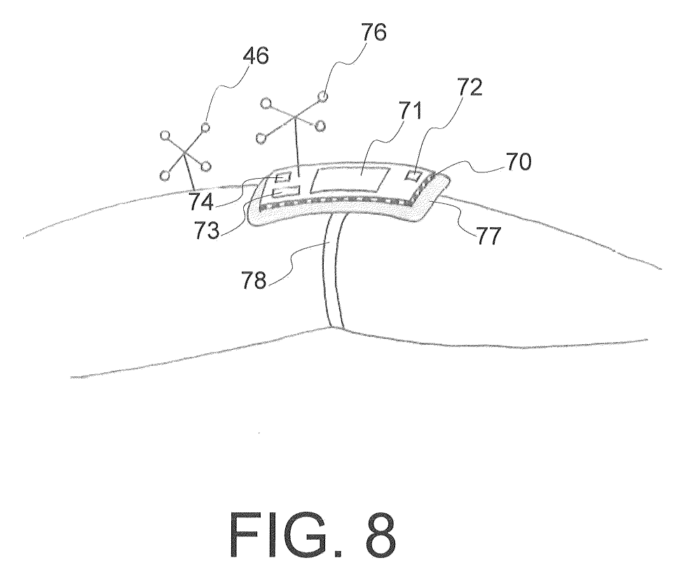

Another embodiment of the transducer array and its use is shown in FIG. 8. As shown, the transducer array 70 is included within a package that also includes a wireless communication module 72, an electronics module 73, and a power module 74. The wireless communication module 72 facilitates wireless communication with a computer via a wireless receiver (not shown). The computer may also be linked to a tracking camera. In the illustrated embodiment, a patient tracking marker 46 and a transducer tracking marker 76 are used to maintain knowledge of the relative positions of the patient anatomy and the transducer array 70, so that the scan acquired by the array is accurately and reproducibly registered with the actual position of the patient anatomy, e.g., in the manner discussed with respect to stage 56 of the flowchart of FIG. 6. In an embodiment, a local display 71 may be provided to show the scan data and/or the model, however this is not required.

An acoustic coupling medium 77 may be disposed between the transducer array 70 and the patient's skin to facilitate reliable transmission and receipt of ultrasound energy. In order to immobilize the transducer array 70 and associated components relative to the patient, a strap 78 may be used.

Although any suitable ultrasound transducer technology such as piezoelectric transducers may be used in implementing the described principles, in an embodiment, captive micromachined ultrasound transducers (CMUTs) are used. These transducers offer fabrication ease for large arrays. A CMUT includes two plate electrodes which are DC biased. One plate is driven with an additional AC signal. In addition to the substrate, the body of each CMUT includes a cavity, a membrane, and the electrode. Other layers may be included as needed, e.g., an insulating layer may also be included to prevent the two electrodes from coming into contact. In an embodiment, a single transducer element may be made up of multiple CMUTs in parallel, with the transducer elements then being arranged to form the desired array.

It will be appreciated that the present disclosure provides a system and method for performing registration of a bone structure to align a computerized model of the bone structure with the actual bone structure. While only certain embodiments have been set forth, alternatives and modifications will be apparent from the above description to those of skill in the art. These and other alternatives are considered equivalents and within the spirit and scope of this disclosure and the appended claims.

What is claimed is:

1. A method for registration of a bone structure of a patient to align a bone structure model with the bone structure, the method comprising:
placing a two-dimensional ultrasound transducer array adjacent the bone structure;
performing an ultrasound scan of the bone structure with the two-dimensional ultrasound transducer array without substantial movement of the two-dimensional ultrasound transducer array relative to the bone structure to identify a surface of the bone structure; and
registering a bone model to the surface of the bone structure.

2. The method for registration of a bone structure of a patient according to claim 1, wherein placing the two-dimensional ultrasound transducer array adjacent the bone structure comprises placing the two-dimensional ultrasound transducer array against a disposable acoustic gel pad situated on the skin of the patient adjacent the bone structure.

3. The method for registration of a bone structure of a patient according to claim 1, further comprising registering a location of the two-dimensional ultrasound transducer array prior to the step of performing an ultrasound scan of the bone structure with the two-dimensional ultrasound transducer array.

4. The method for registration of a bone structure of a patient according to claim 3, wherein registering a location of the two-dimensional ultrasound transducer array comprises registering the location of the two-dimensional ultrasound transducer array relative to a marker attached to the patient.

5. The method for registration of a bone structure of a patient according to claim 3, wherein registering the location of the two-dimensional ultrasound transducer array comprises registering a location of a kinematic mount attached to the two-dimensional ultrasound transducer array.

6. The method for registration of a bone structure of a patient according to claim 5, wherein the kinematic mount is attached to a robotic arm system and wherein registering the location of the kinematic mount comprises registering the location of the kinematic mount by measuring the location of the kinematic mount via the robotic arm system.

7. The method for registration of a bone structure of a patient according to claim 5, wherein registering the location of the kinematic mount comprises registering the location of the kinematic mount by measuring the location of the kinematic mount via one or more inertial sensors.

8. The method for registration of a bone structure of a patient according to claim 1, wherein performing an ultrasound scan of the bone structure with the two-dimensional ultrasound transducer array comprises varying a transmission phase of a plurality of elements of the two-dimensional ultrasound transducer array to steer and focus a beam of acoustic energy.

9. The method for registration of a bone structure of a patient according to claim 1, wherein performing an ultrasound scan of the bone structure with the two-dimensional ultrasound transducer array comprises performing a three-dimensional scan.

10. A system for registration of a bone structure of a patient to align a bone structure model with the bone structure, the system comprising:
a two-dimensional ultrasound transducer array;
a kinematic mount affixed to the two-dimensional ultrasound transducer array; and
a disposable acoustic gel pad interposed between the two-dimensional ultrasound transducer array and the skin of the patient adjacent the bone structure.

11. The system for registration of a bone structure of a patient according to claim 10, further including a processor adapted to execute computer-executable instructions from a nontransitory computer-readable medium, the computer-executable instructions comprising instructions to register the location of the two-dimensional ultrasound transducer array relative to the bone structure.

12. The system for registration of a bone structure of a patient according to claim 11, wherein the instructions to register the location of the two-dimensional ultrasound transducer array include instructions to register the location of the two-dimensional ultrasound transducer array by measuring the location of the kinematic mount relative to the bone structure.

13. The system for registration of a bone structure of a patient according to claim 13, wherein measuring the location of the kinematic mount relative to the bone structure includes measuring the location of the two-dimensional ultrasound transducer array relative to a marker mounted on the patient.

14. The system for registration of a bone structure of a patient according to claim 12, wherein the kinematic mount is attached to a robotic arm system, and wherein the instructions to register the location of the two-dimensional ultrasound transducer array by measuring the location of the kinematic mount includes instructions to measure the location of the kinematic mount via the robotic arm system.

15. The system for registration of a bone structure of a patient according to claim 12, wherein the kinematic mount has associated therewith one or more inertial sensors, and wherein the instructions to register the location of the two-dimensional ultrasound transducer array by measuring the location of the kinematic mount includes instructions to measure the location of the kinematic mount via the one or more inertial sensors.

16. The system for registration of a bone structure of a patient according to claim 11, wherein the computer-executable instructions further comprise instructions to perform an ultrasound scan of the bone structure with the two-dimensional ultrasound transducer array to produce a representation of the bone structure.

17. The system for registration of a bone structure of a patient according to claim 16, wherein the instructions to perform an ultrasound scan of the bone structure with the two-dimensional ultrasound transducer array include instructions to vary a transmission phase of a plurality of elements of the two-dimensional ultrasound transducer array to steer and focus a beam of acoustic energy.

18. The system for registration of a bone structure of a patient according to claim 16, wherein the instructions to perform an ultrasound scan of the bone structure with the two-dimensional ultrasound transducer array include instructions to perform a three-dimensional scan.

19. The system for registration of a bone structure of a patient according to claim 16, wherein the instructions to perform an ultrasound scan of the bone structure with the two-dimensional ultrasound transducer array include instructions to detect any movement of the kinematic mount during the scan.

20. The system for registration of a bone structure of a patient according to claim 19, wherein the instructions to detect any movement of the kinematic mount during the scan include instructions to correct the scan if movement is detected.

21. A non-transitory computer-readable medium having stored thereon computer-executable instructions for registration of a bone structure of a patient via a two-dimensional ultrasound transducer array to align a three-dimensional bone structure model with the bone structure, the computer-executable instructions comprising:
instructions to vary a phase of each of a plurality of elements in the two-dimensional ultrasound transducer array to steer and focus a beam of ultrasound energy;
instructions to scan the focused beam of ultrasound energy in three dimensions through a volume including the bone structure;
instructions to record echo energy while scanning the point focused beam to determine a surface of the bone structure; and
instructions to align a model of the bone structure with the determined surface of the bone structure.
